(19) United States
(12) Patent Application Publication
(10) Pub. No.: US 2022/0040265 A1
(43) Pub. Date: Feb. 10, 2022

(54) COMPOSITIONS AND METHODS FOR PREVENTING AND/OR INHIBITING VIRAL INFECTION AND SPREAD

(71) Applicant: Peter G. Carroll, Canton, MA (US)

(72) Inventor: Peter G. Carroll, Canton, MA (US)

(21) Appl. No.: 17/394,854

(22) Filed: Aug. 5, 2021

Related U.S. Application Data
(60) Provisional application No. 63/086,427, filed on Oct. 1, 2020.
(60) Provisional application No. 63/076,103, filed on Sep. 9, 2020.
(60) Provisional application No. 63/062,726, filed on Aug. 7, 2020.

(51) Int. Cl. A61K 38/21; A61K 9/00

(52) U.S. Cl. CPC A61K 38/212; A61K 9/0073; A61K 38/217; A61K 38/215

(57) ABSTRACT

Use of an interferon (INF), whether Type I (e.g. interferon-alpha, interferon-beta, etc.), Type II (e.g. interferon-gamma) and/or Type III (e.g. interferon-lamda), for inhibiting and/or preventing coronavirus infection in people, including but not limited to those people at risk of exposure to COVID 19 and/or variants thereof.

### TECHNICAL FIELD

The present invention relates to new use of an interferon (INF), whether Type I (e.g. interferon-alpha, interferon-beta, etc.), Type II (e.g. interferon-gamma) and/or Type III (e.g. interferon-lamda), for inhibiting and/or preventing coronavirus infection in people, including but not limited to those people at risk of exposure. This includes, but is not limited to, the elderly, people in nursing homes and hospitals (both patients and workers), students and teachers, members of team sports, people exposed to small or large crowds (e.g. spectators at an event, people traveling on planes, etc.), and people in locations where infections are high and/or increasing (e.g. particular cities, states or countries).

### BACKGROUND

Coronaviruses (CoV) are a large family of viruses that cause illness ranging from the common cold to more severe diseases such as Middle East Respiratory Syndrome (MERS-CoV) and Severe Acute Respiratory Syndrome (SARS-CoV). A novel coronavirus (nCoV) is a new strain, recently named Covid-19 by the World Health Organization, that had not been previously identified in humans. It is estimated that millions have been infected with Covid-19 and hundreds of thousands have died (and the pandemic is by no means over).

Coronaviruses are zoonotic, meaning they are transmitted between animals and humans. Detailed investigations found that SARS-CoV was transmitted from civet cats to humans and MERS-CoV from dromedary camels to humans. COVID-19 is believed to have come from bats. Several known coronaviruses are circulating in animals that have not yet infected humans. Thus, there is reason to believe that there are more coronaviruses (beyond COVID-19) that pose a threat to human health in the future.

The majority of COVID-19 patients infected with the virus experience mild flu-like symptoms. However, a significant minority experience moderate to severe respiratory symptoms, including shortness of breath and impaired oxygen saturation. These patients typically require hospitalization, and progress to being intubated and/or ventilator dependent. The percentage of COVID-19 patients who require hospitalization, and progress to being intubated and/or ventilator dependence climbs significantly with age, the presence of underlying diseases, the presence of secondary infection and elevated inflammatory indicators in the blood.

This is not to say that children do not get infected or do not spread the virus. Indeed, a recent article in the journal JAMA reports findings from a pediatric hospital in Chicago, Ill. The Chicago study examines the concentration of the SARS-CoV-2 in the nasopharynx, or the upper region of the throat that connects to the nasal passages, of children and adults. According to the results, children 5 years and younger who develop mild to moderate Covid-19 symptoms have 10 to 100 times as much SARS-CoV-2 in the nasopharynx as older children and adults.

Moreover, infection in children appears to be greater in minorities. A study published in the journal Pediatrics examined 1,000 patients tested at a Children's National Covid-19 testing site in Washington, D.C., between March 21 and April 28. Of that group, just 7.3% of White children tested positive for coronavirus, in contrast to 30% of Black children and 46.4% of Hispanic children.

Fatalities appear to be highest in the elderly, ranging from 3% to 27%, among persons aged 65-84 years, respectively. Nursing homes have been a target for the virus. Although people who live in nursing homes make up only one percent of the U.S. population, they account for 43 percent of the U.S. coronavirus deaths in the first wave. As variants have immerged, such as the Alpha and Delta variants (discussed more below), younger people now face increasing risk of hospitalization and death.

So-called “social distancing” (where people stay approximately six feet apart) and masks have slowed the spread of COVID-19. But social distancing is difficult in many situations and compliance with masks has proven to be a problem. Some essential industries, for example, have processing facilities where workers are in close contact. The meat processing industry in particular has been hard hit by the virus. At least 49,369 U.S. meatpacking, food processing and farmworkers have contracted Covid-19 since March of 2020, over 10,000 of whom were meatpackers at Tyson foods, whose brands include Jimmy Dean, Hillshire Farm and Sara Lee. In Brazil, JBS SA, the world's largest meatpacker has reported outbreaks in 23 plants, with 4,000 JBS employees testing positive for coronavirus and at least six dying from COVID-19.

Cruise ships are another place where social distancing is difficult. As a result, the cruise ships were hit relatively hard with infections and the industry had to shut down operations. The Norwegian cruise line Hurtigruten was the first cruise operator worldwide to return an oceangoing cruise ship to service in mid-June 2020, touting reduced passenger capacity, social distancing and strict rules on hygiene. But a large infection in early August of 2020 has caused it to suspend all so-called expedition cruises until further notice.

The American Navy has also been victimized by Covid-19. Again, this is a situation where social distancing is difficult to impossible. While an aircraft carrier is the most spacious ship in the fleet, the more than 5,000 sailors work and sleep in small enclosed areas. The American aircraft carrier USS Theodore Roosevelt was one of the first instances where the virus struck a deployed American naval ship. The Navy report that, in May of 2020, 1,156 crewmembers had tested positive, with one fatality. In response, the Navy ordered a stop to all port visits to reduce the chance of spreading the virus through the fleet. The aircraft carrier Dwight D. Eisenhower and the cruiser San Jacinto were ordered to keep moving and avoid all port visits. As a result, they notched their 161st consecutive day at sea, breaking the previous Navy record of 160 days.

Schools are another place where social distancing is difficult if teaching is done in person. Indeed, the start of the new school year is already proving problematic. While preparing for the start of the year, five employees in the Marietta City Schools system in Georgia tested positive for coronavirus. And on the first day of school in Canton, Ga., a second grader tested positive, sending the teacher and 20 other classmates into a two-week quarantine.

As a result of the rapid spread of the virus, cities and states have imposed so-called “lockdowns,” where people are restricted to essential movement, and high trafficked establishments such as schools, restaurants, bars, theaters, sports venues, gyms and the like are closed. The results of such lockdowns on the number of infections have been good, as seen in the decline in infections in Massachusetts and New York City. However, the economic impact of these closings is high. Small business, including restaurants and bars, have been hit so hard that some have closed permanently. Large retail chains have also suffered, with many top name companies shutting their doors. Airlines have had to cut their schedules and furlough employees. Even amusement parks have suffered. Disney's theme park closures have had a significant impact on the company's finances, with the parks division reporting a $2 billion loss in its third quarter due to the ongoing closures. While Disney furloughed employees early on, by October of 2020 they were forced to lay off 28,000 employees.

Indeed, the economic impact has caused some states to lift their lockdowns. Unfortunately, by lifting the lockdowns early, some states have seen the spread of the infection go unchecked. Some states that lifted the lockdowns too early, like California, have been forced to re-establish them, providing a second blow to unemployment and economic growth. However, a number of southern states refuse to re-establish the lockdowns. Given the aggressive rate of spread of COVID-19, significant concern exists that the US healthcare system does not have the number of ventilators and/or ICU beds to meet the expected demand in the coming months. This has threatened to overwhelm the health care system. In Florida, at least 54 hospitals have reached capacity in their intensive care units and show zero ICU beds available at the end of July 2020, according to data released by the Agency for Health Care Administration (AHCA). Ten of the hospitals at capacity are in Miami-Dade County, and eight of them are in Broward County, AHCA data shows. Another 44 hospitals have 10% or less ICU capacity available, according to AHCA. AHCA reports about 16% ICU beds are available across the State of Florida. This creates the risk of not being able to treat infected patients properly. It also increases the risk of infection for hospital doctors, nurses and staff. Indeed, more than 3,600 U.S. health care workers perished in the first year of the pandemic. While Florida handled this crisis in 2020, the number of new daily cases on July 31 of 2021 in Florida was 21,683, the state's highest one-day total since the start of the pandemic.

More testing has also been proposed. However, even with testing, several colleges have had outbreaks. For example, the University of North Carolina suspended sports when 37 athletes, coaches and staff tested positive. As the Fall of 2020 approaches, colleges planning on testing all incoming students are finding that testing capabilities around the country are extremely strained. Indeed, some colleges are canceling in-person class because of the inability to get adequate testing.

Other colleges have attempted to proceed with students on campus, but this experiment appears to be failing badly. The University of Notre Dame in August of 2020 announced it would switch to remote learning, after welcoming students back to campus for the start of the fall semester just one week earlier. As with other colleges, coronavirus cases rose sharply days after Notre Dame's reopening, with 146 students testing positive. By Sep. 1, 2020, more than 20,000 cases of Covid-19 among students and staff have been reported at colleges and universities across at least 36 states.

Sports teams have in some cases decided to play regular games, opting to play in empty stadiums. But the lack of fans in the stands has not made players safe. As the major league baseball season started up in 2020, the Miami Marlins had 17 players test positive in the first five days. Then, two staff members of the Philadelphia Phillies staff tested positive for coronavirus days after the team played the Miami Marlins. Just days later, it was announced that members of the St. Louis Cardinals have tested positive. Based on this, it is reasonable to predict that other teams and athletes are at risk. Indeed, Commissioner Rob Manfred reportedly told the Players Association that if players don't do a better job managing outbreaks, the season would be canceled. Moreover, baseball is not the only sport where this has happened. After the start of the 2020 NFL season, it was reported that a number of Titans players tested positive for Covid-19, forcing the Titans and the Minnesota Vikings, who played the Titans, to suspend activities and postpone upcoming games.

Vaccines are under development and some have been approved for emergency use. Nonetheless, it remains to be seen whether protective immunity can be generated safely with a vaccine to COVID-19, as well as downstream variants caused by mutation. There is the concern as to whether the antibodies raised will only last a short time. There is a further concern that the companies pursuing a vaccine are cutting corners in favor of speed. There is also the concern (as with all vaccines) of rare side effects only detected after giving it to thousands of people (or more). For example, Pandemrix, a vaccine for swine flu, was given to around six million people in the UK in 2009-10. It was found to cause narcolepsy, a debilitating sleep disorder, in around one in every 55,000 vaccinated individuals in the UK, or just over 100 people. A further example is RotaShield, a vaccine against diarrhea-causing rotaviruses released on the American market in the late 1990s. The vaccine was linked to a very small number of cases of intussusception, a life-threatening intestinal disorder, and was rapidly withdrawn from use. A replacement was not available for close to a decade. Finally, there is the concern about the number of people who will be willing to take the vaccine; reports indicate a large percentage of Americans are reluctant to get the vaccine. Indeed, as of mid-July of 2021, only 48.2% of the US population is fully vaccinated (160,126,516 people). Only 20 states have fully vaccinated more than half of their residents. This is having a significant negative impact. Over 90% of those Americans who have tested positive or been admitted to the hospital in the summer of 2021 are unvaccinated people. In states like Missouri, hospitals are now busier in July of 2021 than at any previous point during the pandemic. In just five weeks, some hospitals took in as many COVID-19 patients as it did over five months in 2020.

There is also the specific concern of antibody dependent enhancement (ADE) with a vaccine that induces antibodies. ADE is where the antibody raised by a vaccine serves to enhance (rather than inhibit) viral entry into cells. See Wan et al., “Molecular Mechanism for Antibody-Dependent Enhancement of Coronavirus Entry,” J. Virology 2020 Feb. 14; 94 (5). In short, ADE would make the vaccinated person more at risk (or worse).

Drugs are also being tested to treat infected patients. But this is an “after the fact” approach that does not address prevention.

What is needed is a way to inhibit or prevent viral infection and spread that would allow for the lifting of lockdowns at least in part, e.g. allowing for people to go to school, restaurants and sporting events.

### SUMMARY OF THE INVENTION

The present invention relates to new use of an interferon (INF), whether Type I (e.g. interferon-alpha, interferon-beta, etc.), Type II (e.g. interferon-gamma) and/or Type III (e.g. interferon-lamda), for inhibiting and/or preventing coronavirus infection in people including but not limited to those people at risk of exposure, including those at risk of exposure to variants such as the Alpha and Delta variants (discussed below). In one embodiment, a person is at risk of exposure where their job brings them into contact with people who have tested positive for the virus, e.g. nurses, doctors in hospitals where people who have tested positive are admitted. In one embodiment, a person is at risk of exposure where their job brings them into contact with people who are not vaccinated, or not fully vaccinated. As of July of 2021, the majority of COVID patients in hospitals are not vaccinated. Many have the Delta variant. Thus, in one embodiment, the present invention contemplates giving hospital workers—both vaccinated and unvaccinated—interferon. Thus, in one embodiment, the present invention contemplates administering interferon to hospital workers where said hospital has patients who have tested positive for the Delta variant of COVID-19. The present invention contemplates administering an interferon (or combinations of different interferons) through inhalation via an aerosol, spray, mist, or powder that patients and employees inhale into their airways (including but not limited to, providing the necessary materials to allow people to self-administer interferon). This includes, but is not limited to, i) the elderly, ii) patients and workers in nursing homes, hospitals, clinics and doctor offices, iii) students and faculty (e.g. high school, college, etc.), iv) workers in close contact (including those involved in meatpacking and food processing, as well as those in the military, such as those on naval ships), v) members of team sports (including the coaches and staff), vi) people exposed to groups and crowds (e.g. spectators at an event, audiences in a theater, people traveling, restaurant and bar staff, amusement park employees, theme park employees, nature park employees (including zoo employees) etc.), and vii) people in locations where infections are high and/or increasing (e.g. in particular cities, states or countries, such as Alabama, Missouri, Tennessee and the like). In one embodiment, the people to whom interferon is administered tested negative for COVID-19 infection. By inhibiting infection in individuals, it is contemplated that spread of the virus throughout the population will also be reduced.

In one aspect of the invention, there is administration of one or more agents selected from: (i) IFN-alpha, ii) INF-beta iii) INF-gamma and iv) IFN-lamda and (v) agents that increase IFN expression (as well as combinations thereof). In one embodiment, administration to the airway in individuals is preferred. In one embodiment, nasal (including bilateral administration, i.e. both nostrils) or oral administration is preferred. The nasal cavity is covered by a thin mucosa. Without being bound by theory, it is believed that exposure of this mucosa (and the associated immune cells) to an interferon will generate protection against coronavirus infection, including but not limited to Covid-19 and variants thereof. In one embodiment, the mucosal immune system is activated by aerosolized interferon as a defense against infection.

It is believed that a person with an activated mucosal immune system will be better positioned to fight off the virus. This added defense against the coronavirus (as well as other viruses) is likely to last for a few days after a single administration of interferon. Therefore, in one embodiment, the present invention contemplates an additional administration if the person is going to be at risk (e.g. potentially exposed again to the virus) days after the first administration. Nonetheless, where the person is going to an event lasting a few hours, e.g. a sports or theater event, it is contemplated that the single administration of interferon prior to the event will provide an added defense against virus infection for the duration of the event. Similarly, in one embodiment, it is contemplated that the athletes, coaches and staff will be provided a single administration of interferon as described herein for protection during the game. Again, in one embodiment, interferon is administered to these people even though they have tested negative or would test negative if tested for the virus.

Mouse studies involving vesicular stomatitis virus involved continuous exposure to aerosols of interferon for 8 hours. See “Duration of Effect of Interferon Aerosol Prophylaxis in Vesicular Stomatitis Virus Infection in Mice,” Wyde et al., Antimicrobial Agents and Chemotherapy 27:60 1985). By contrast, the present invention contemplates a single administration that can be performed completely in 1 minute or less, and more preferably 30 seconds or less, and still more preferably in 10 seconds or less.

In some embodiments, it is contemplated that administering aerosolized interferon through the nasal or oral cavity will result is some deposition of interferon in the lung. Since COVID-19 primarily attacks the lungs, this is an added benefit. Indeed, in one embodiment, administration is done to ensure deposition in the lung. Importantly, delivery of the medication through the oral or nasal route largely avoids exposure to other organ systems, i.e. organs other than the lung. Thus, any significant systemic exposure is avoided.

In one aspect, a method of preventing and/or inhibiting coronavirus infection and spread comprises administering to an individual in need thereof, such as a person at risk of exposure to the virus, an aerosol, spray, mist or powder comprising an pharmaceutical composition containing an amount of an interferon (or combination of interferons) effective to inhibit and/or prevention infection and spread. In one example, the coronavirus is Covid-19. In one embodiment, the person is tested for infection prior to administering interferon. In one embodiment, interferon is administered to these people even though they have tested negative or would test negative if tested for the virus. In one embodiment, the person's temperature is taken prior to such administering. In one embodiment, the person is given the necessary materials for self-administration. After all, people with asthma learn how to administer inhaled medications themselves.

Speaking of asthma, the present invention contemplates administration of aerosolized interferon to people with background health conditions that make them more susceptible to infections and/or more likely to have more serious outcomes after infection. People with respiratory health conditions, such as asthma, can be administered aerosolized interferon to provide an added defense against a coronavirus, and in particular COVID-19. The CDC has indicated people with the following conditions, in addition to those with moderate to severe asthma, may be at an increased risk for severe illness from COVID-19: Cerebrovascular disease (affects blood vessels and blood supply to the brain), Cystic fibrosis, Hypertension or high blood pressure, Immunocompromised state (weakened immune system) from blood or bone marrow transplant, immune deficiencies, HIV, use of corticosteroids, or use of other immune weakening medicines, Neurologic conditions, such as dementia, Liver disease, Pregnancy, Pulmonary fibrosis (having damaged or scarred lung tissues), Smoking, Thalassemia (a type of blood disorder), Type 1 diabetes mellitus. Thus, the present invention contemplates administering aerosolized interferon to people with these conditions to inhibit and/or prevent infection, as well as lessen the severity of symptoms should such a person become infected after aerosolized administration of interferon. Again, in one embodiment, interferon is administered to these people even though they have tested negative or would test negative if tested for the virus.

In addition, the CDC has indicated that people of any age with the following conditions are at increased risk of severe illness from COVID-19: Cancer, Chronic kidney disease, COPD (chronic obstructive pulmonary disease), Immunocompromised state (weakened immune system) from solid organ transplant, Obesity (body mass index [BMI] of 30 or higher), Serious heart conditions, such as heart failure, coronary artery disease, or cardiomyopathies, Sickle cell disease, and Type 2 diabetes mellitus. Thus, the present invention contemplates administering aerosolized interferon to at risk people with these conditions to inhibit and/or prevent infection, as well as lessen the severity of symptoms should such a person become infected after aerosolized administration of interferon. Again, in one embodiment, interferon is administered to these people even though they have tested negative or would test negative if tested for the virus.

It is not necessary that infection is 100% inhibited or prevented in every individual who is administered aerosolized interferon in accordance with the present invention. It is sufficient that people administered aerosolized interferon as a group have fewer instances of infection than a group of people not administered. It is also expected that, if a person administered aerosolized interferon does become infected, their symptoms will be more mild and their recovery will be achieved more quickly (again, as compared to people not administered aerosolized interferon).

In a preferred embodiment, administration of interferon as described herein is not meant to be the only defense used against coronaviruses, and in particular COVID-19. In a preferred embodiment, it is meant to be additive, i.e. one more layer of defense. Hospital workers and others in high risk occupations should continue to wear Personal Protective Equipment (PPE) whether or not they receive interferon as described herein. This includes (but is not limited to) wearing 1) eye protection in addition to their facemask to ensure the eyes, nose, and mouth are all protected from exposure to respiratory secretions during patient care encounters; and 2) a N95 or equivalent or higher-level respirator, instead of just a cloth facemask.

In one embodiment, the present invention contemplates the use of an agent selected from Type I (e.g. interferon-alpha, interferon-beta, etc.), Type II (e.g. interferon-gamma), Type III (e.g. interferon-lamda) and combinations thereof, in the manufacture of a medicament for administration to inhibit and/or preventing coronavirus infection in people, including but not limited to those people at risk of exposure to a coronavirus, including but not limited to COVID-19 and variants thereof. In one embodiment, the person has a respiratory disorder. In one embodiment, said respiratory disorder is asthma. In one embodiment, the use is via airway delivery. In one embodiment, it is nasal (including bilateral) or oral administration.

In one embodiment, the present invention contemplates a method of administering aerosolized interferon to a person at risk of exposure to a coronavirus, including but not limited to COVID-19, comprising administering to the person's airways (whether through nasal or oral routes) an amount of an agent selected from Type I (e.g. interferon-alpha, interferon-beta, etc.), Type II (e.g. interferon-gamma), Type III (e.g. interferon-lamda) and combinations thereof.

In one embodiment, the present invention contemplates a method of inhibiting coronavirus infection (including but not limited to COVID-19) comprising administering interferon to at least a portion of the upper respiratory tract (e.g. nasal tract, oral tract, etc.) of a person at risk of being exposed to a coronavirus. In one embodiment, said interferon is selected from the group consisting of a Type I interferon, a Type II interferon, a Type III interferon, or combinations thereof. In one embodiment, said interferon is IFN-alpha. In one embodiment, said interferon is IFN-beta. In one embodiment, said interferon is IFN-lambda. In one embodiment, said interferon is IFN-gamma. In one embodiment, said administering is through inhalation of an aerosol, spray, mist, or powder comprising said interferon. In one embodiment, said administering is done by a health professional. In one embodiment, said administering is self-administration. In one embodiment, said person is elderly (e.g. 55 or older). In one embodiment, the person is a patient or worker in a nursing home. In one embodiment, the person is a patient in a doctor or dentist office (or the dentist). In one embodiment, the person administered interferon is going to a hospital for treatment that is unrelated to COVID-19, but is at risk of exposure from the COVID-19 infected patients in the hospital. In one embodiment, the person is a student or teacher in a school. In one embodiment, the person is a worker in a meatpacking and/or food processing facility. In one embodiment, the person is a member of a sports team. In one embodiment, the person is an actor in a theater. In one embodiment, the person is a worker in a restaurant or bar. In one embodiment, the person is a transportation worker. In one embodiment, the person is spectator at an event. In one embodiment, the person works at or plans to enter an amusement park or theme park. In one embodiment, interferon is administered to these people even though they have tested negative or would test negative if tested for the virus. In one embodiment, each person administered interferon via inhalation tested negative for COVID-19 infection. In one embodiment, more than one type of interferon is administered simultaneously, separately or sequentially. In one embodiment, the administration results in activation of at least a portion of the immune cells in said portion of said upper respiratory tract. In one embodiment, the interferon is administered by means of an aerosol nebulizer. In one embodiment, the interferon is administered by pressurized container, pump, spray or nebulizer. In one embodiment, between 1 and 1,000 micrograms of interferon is administered in a single administration. In one embodiment, said single administration takes 1 minute or less (and preferably 30 seconds or less).

In yet another embodiment, the present invention contemplates a method for inhibiting viral infection of a person, comprising administering 1 to 1,000 micrograms of an interferon to said person via inhalation prior to close contact with multiple people (3 or more people, more typically 10 or more people) or crowds (30 or more people, more typically 100 or more people, still more typically 1000 or more people). In one embodiment, said interferon is selected from the group consisting of a Type I interferon, a Type II interferon, a Type III interferon, or combinations thereof. In one embodiment, said administering is through inhalation of an aerosol, spray, mist, or powder comprising said interferon. In one embodiment, said person is a member of a sports team and said aerosolized interferon is administered prior to (e.g. 2 days prior to, 1 day prior to, less than 24 hours prior to, less than 12 hours prior to, less than 3 hours prior to, or less than 60 minutes prior to) practice or a game with other members of a sports team (whether high school, college or professional sports). In one embodiment, the person administered interferon by inhalation tested negative for COVID-19 infection. In one embodiment, said interferon is aerosolized. In one embodiment, said aerosolized interferon contacts at least the nasal mucosa of said person. In one embodiment, said viral infection is a coronavirus infection. In one embodiment, said coronavirus is COVID-19 or variant thereof. In one embodiment, a single administration of less than 100 ug of aerosolized interferon is administered using a nebulizer. In one embodiment, said administering results in a portion of said aerosolized interferon depositing in the lungs of said person. In one embodiment, interferon is administered to these people even though they have tested negative or would test negative if tested for the virus.

In yet another embodiment, the present invention contemplates a method for inhibiting viral infection of a sports team, comprising administering 1 to 1,000 micrograms of an interferon to at least all of the participating members of a first sports team via inhalation prior to (e.g. 2 days prior to, 1 day prior to, less than 24 hours prior to, less than 12 hours prior to, less than 3 hours prior to, or less than 60 minutes prior to) practice or a game. By participating members, it is meant those members who will actually play in the practice or game, as opposed to those who will sit out the practice or game. For example, it is common that only a portion of a team will participate in a particular game (e.g. only one or a handful of pitchers typically participate in a baseball game). It is intended that the administration be at least to that portion. However, it is not intended that the present invention be limited to administration of that portion, since non-participants may be sitting together or coming in contact with participants. Thus, in a preferred embodiment, both participating and non-participating members of the sports team are administered interferon. In one embodiment, all coaches and staff are similarly administered interferon. In one embodiment, this is done where the game is not played in front of spectators, e.g. fans in the stands. In one embodiment, said interferon is selected from the group consisting of a Type I interferon, a Type II interferon, a Type III interferon, or combinations thereof. In one embodiment, said administering is through inhalation of an aerosol, spray, mist, or powder comprising said interferon. In one embodiment, the method further comprises administering 1 to 1,000 micrograms of an interferon to at least all of the participating members of a second sports team via inhalation prior to (e.g. 2 days prior to, 1 day prior to, less than 24 hours prior to, less than 12 hours prior to, less than 3 hours prior to, or less than 60 minutes prior to) practice or a game with said first team. In this embodiment, first and second football, baseball, hockey, soccer and track teams will be administered in advance of the practice or game. In one embodiment, said interferon is aerosolized. In one embodiment, said aerosolized interferon contacts at least the nasal mucosa of said participating members. In one embodiment, said viral infection is a coronavirus infection. In one embodiment, said coronavirus is COVID-19 or variant thereof. In one embodiment, a single administration of less than 100 ug (but more than 0.1 ug) of aerosolized interferon is administered using a nebulizer to each participating member of said first team. In one embodiment, said sports team is a football team. In one embodiment, said sports team is a baseball team. In one embodiment, said sports team is a hockey team. In one embodiment, said sports team is a soccer team. In one embodiment, said sports team is a track team. In one embodiment, the sports team is selected from the group consisting of rugby, boxing, golf, snooker, horse racing and the equestrian sports teams. In one embodiment, the team is a Gaelic Football team or a Hurling team. In one embodiment, the team is badminton, billiards, bowls, boxing, cricket, croquet, curling, darts, golf, fives, hockey, netball, rugby, tennis, table tennis, snooker, squash, or water polo team. In one embodiment, the team is a rowing, dance sports or motor sports team. In one embodiment, no participating member of said first team is known to be infected with a coronavirus (e.g. known to have symptoms or to have tested positive). In one embodiment, interferon is administered to these people even though they have tested negative or would test negative if tested for the virus. In one embodiment, all of the participating members of said first team have all tested negative for COVID-19 infection.

In yet a further embodiment, the present invention contemplates a method for inhibiting viral infection of spectators of a sporting event at a sporting venue, comprising administering 1 to 1,000 micrograms of an interferon via inhalation to each person entering the sporting venue at an entry point (e.g. any type of entrance, door or gateway), prior to said sporting event. In one embodiment, said administering is done at a site around (or in) the venue, whether a booth, tent, stall, stand, kiosk, table, window or counter. In one embodiment, said interferon is administered through inhalation via an aerosol spray, mist, or powder. In one embodiment, said entry point comprises a turnstile and said administering takes place at or near the turnstile. In one embodiment, said administering takes place just prior to the person coming through the turnstile. In one embodiment, said administering takes place as the person is coming through the turnstile. In one embodiment, said administering takes place just after the person has come through the turnstile. In one embodiment, the method further comprises the step of adding turnstiles (or other type of gateway) to the sports venue (e.g. stadium, arena, etc.) in advance of the sporting event (e.g. in order to accommodate this additional time needed for interferon administration to the spectators entering the venue). In one embodiment, interferon is administered to these people even though they (or a portion thereof) have tested negative or would test negative if tested for the virus.

As of Jul. 21, 2021, there have been a total of almost 206,000 COVID-19 cases within the military. Navy sailors account for more than 39,700 of those cases. In a further embodiment, the present invention contemplates administering an interferon (or combinations of different interferons) through inhalation via an aerosol, spray, mist, or powder that military persons inhale into their airways (including but not limited to, providing the necessary materials to allow people to self-administer interferon). This includes, but is not limited to, military who are deployed, such as those on naval ships. As noted above, after the aircraft carrier USS Theodore Roosevelt reported crewmembers testing positive for Covid-19, the Navy ordered a stop to all port visits to reduce the chance of spreading the virus through the fleet. This meant that sailors would not get shore leave. But this problem can be remedied. In one embodiment, the present invention contemplates administering 0.1 to 1000 micrograms, and more typically 1 to 1,000 micrograms, of an interferon via inhalation to each military person on board a ship, and/or to each military person taking shore leave. In one embodiment, a single administration of less than 100 ug (but more than 0.1 ug) of aerosolized interferon is administered using a nebulizer to each sailor, officer or staff on board a ship, and/or to each person taking shore leave. By giving to those taking shore leave, a measure of protection is given to them against infection from infected people contacted in port. By giving it to all of those on board, a measure of protection is given to everyone, including protection for those that did not take shore leave but are at risk of infection from those who did take shore leave (once they return to the ship). Thus, in one embodiment, the present invention contemplates a method for inhibiting viral infection (including but not limited to COVID-19) of members of the military, comprising administering 1 to 1,000 micrograms of an interferon via inhalation to said military person. In one embodiment, said interferon is administered through inhalation via an aerosol spray, mist, or powder. In one embodiment, said administering is on a military ship or military supply ship. In one embodiment, said administering takes place just prior (e.g. minutes to hours prior) to when the military person takes shore leave at a port of call (or as they depart the ship). In one embodiment, said administering takes place at least 8 hours (and up to 2 days) prior to when the military person takes shore leave at a port of call. In one embodiment, said administering to a military person is done just prior to being deployed (e.g. prior to joining a crew on a ship). The administering can be combined with testing and/or taking the person's temperature, e.g. after testing negative for COVID-19. In one embodiment, interferon is administered to these military people even though they (or a portion thereof) have tested negative or would test negative if tested for the virus.

Military recruits are prone to outbreaks of respiratory tract infections due to their crowded living conditions in barracks. Once again, it is a situation where social distancing is not practical. In one embodiment, the present invention contemplates administering an interferon (INF), whether Type I (e.g. interferon-alpha, interferon-beta, etc.), Type II (e.g. interferon-gamma) and/or Type III (e.g. interferon-lamda), for inhibiting and/or preventing coronavirus infection in military people, including recruits in barracks, through inhalation via an aerosol, spray, mist, or powder that they inhale into their airways (including but not limited to, providing the necessary materials to allow military people to self-administer interferon). In a preferred embodiment, low-dose recombinant human interferon a-2b (rIFNa-2b) is administered as a nasal spray, e.g. a device is used to deliver 0.1-0.3 ml (3-9×10⁵ IU of rIFNa-2b) per spray into each nostril and throat, such that a total of 9-27×10⁵ IU of rIFNa-2b are administered. The spray, mist or aerosol can be delivered once or twice daily, e.g. after breakfast and supper, for a number of consecutive days (3, 4, 5, 6, 7 days or more). Alternatively, it can be given every other day or even every third day. In one embodiment, interferon is administered to these people even though they (or a portion thereof) have tested negative or would test negative if tested for the virus.

In one embodiment, the present invention contemplates administering an interferon (INF), whether Type I (e.g. interferon-alpha, interferon-beta, etc.), Type II (e.g. interferon-gamma) and/or Type III (e.g. interferon-lamda), for inhibiting and/or preventing coronavirus in vaccinated individuals. In one embodiment, the present invention contemplates administering an interferon (INF), whether Type I (e.g. interferon-alpha, interferon-beta, etc.), Type II (e.g. interferon-gamma) and/or Type III (e.g. interferon-lamda), for inhibiting and/or preventing coronavirus in unvaccinated individuals, including those who refuse vaccination.

### DESCRIPTION OF THE INVENTION

The present invention relates to new use of an interferon (INF), whether Type I (e.g. interferon-alpha, interferon-beta, etc.), Type II (e.g. interferon-gamma) and/or Type III (e.g. interferon-lamda), for inhibiting and/or preventing coronavirus infection in people including but not limited to those people at risk of exposure to virus such as a coronavirus. The present invention contemplates administering an interferon (or combinations of different interferons) through inhalation via an aerosol, spray, mist, or powder that patients inhale into their airways (including but not limited to, providing the necessary materials to allow people to self-administer interferon). This includes, but is not limited to, i) the elderly, ii) patients and workers in nursing homes, hospitals, clinics and doctor offices, iii) students and faculty (e.g. high school, college, etc.), iv) workers in close contact (including those involved in meatpacking and food processing), v) members of team sports (including the coaches and staff), vi) people exposed to groups and crowds (e.g. spectators at an event, audiences in a theater, people traveling, restaurant and bar staff, amusement park employees etc.), and vii) people in locations where infections are high and/or increasing (e.g. in particular cities, states or countries, such as those where the incidence of vaccination is less than 70%, more typically less than 60%, still more typically less than 50%, and including 40% or less). By inhibiting infection in individuals, it is contemplated that spread of the virus throughout the population will also be reduced. In one embodiment, interferon is administered to these people even though they have tested negative or would test negative if tested for the virus.

It is not intended that the present invention be limited to the type of person for administration of aerosolized interferon. In a preferred embodiment, the person is at risk of being exposed to virus. In one embodiment, the present invention contemplates aerosolized interferon administration to so-called first responders, including but not limited to, nurses, doctors, hospital staff, police, and fire department personnel. In another embodiment, the present invention contemplates aerosolized interferon administration to i) the elderly, ii) patients and workers in nursing homes, hospitals, clinics and doctor offices, and iii) students and faculty. In another embodiment, the present invention contemplates aerosolized interferon administration to people who regularly come in contact with strangers as part of their job, including but not limited to restaurant and bar staff, grocery workers, transportation workers (mass transit workers, bus and train employees, cab drivers, delivery people, pilots and flight attendants, cruise ship workers, etc.) and entertainers (including the members and staff of sport teams, theater actors and staff, etc.). In yet another embodiment, the present invention contemplates aerosolized interferon administration to people prior to (or shortly thereafter) entering a structure, vehicle or situation involving close contact (e.g. less than six feet apart, and typically less than three feet apart, and more typically less than one foot apart) with multiple people or crowds (e.g. more than 10 people, and typically more than 20 people, and more typically 30 people or more), including but not limited to a sporting event, a movie or theater event, travel (whether on plane, train, ship, boat or bus), or school (e.g. class room, lecture hall, dorm room, etc.). In one embodiment, interferon is administered to these people even though they have tested negative or would test negative if tested for the virus.

In one embodiment, the present invention contemplates aerosolized interferon administration to people entering a hospital where patients are being treated for COVID-19 infections. For example, a person may enter a hospital for treatment that is unrelated to COVID-19, but be at risk of exposure from the COVID-19 infected patients in the hospital. By administering (or self-administering) a preventative dose of aerosolized interferon, it is contemplated that such an in-coming person will have added defense in the hospital (or doctor's office) against infection.

In a preferred embodiment, the present invention contemplates administering interferon via an aerosol, spray, mist, or powder that patients inhale into their airways (including but not limited to, providing the necessary materials to allow people to self-administer interferon), in cases where the person is going to be performing a task on another person who might be infected. For example, dentists and their assistants, work on people who have their mouths open. Since access to the person's teeth is a must, the patient cannot wear a mask through the entire visit to the dentist. In such a case, interferon administration to dentists and their assistants may provide an extra layer of defense. Moreover, dentist offices may ask their patients to allow for interferon administration before a procedure. Again, this may provide an extra layer of defense. While dentists have been used as an example, it is not intended that the present invention be limited thereto. Salons (e.g. hair and nail), barbershops, physical therapy facilities and the like all will benefit from the same approach. In one embodiment, interferon is administered to these people even though they have tested negative or would test negative if tested for the virus.

One particularly preferred embodiment involves sport events and sport venues. The present invention contemplates, in one embodiment, allowing for team sports by first administering to the team itself (or at least to the participating members). For example, Major League Baseball is currently playing games without fans in the stands. Nonetheless, as described above, infections among the athletes and staff are threatening the (already shortened) 2020 season. The present invention contemplates administering interferon via inhalation to the athletes and staff prior to (e.g. 2 days prior to, 1 day prior to, less than 24 hours prior to, less than 12 hours prior to, less than 3 hours prior to, or less than 60 minutes prior to) a baseball game, or game in another sport (including but not limited to those listed above in the Summary). In a preferred embodiment, this includes the athletes and staff of both teams, as well as any official (e.g. umpires, referees, etc.) and camera (or other equipment) operators. In one embodiment, this is done where the game is not played in front of spectators, e.g. fans in the stands. In another embodiment, this is done where there are spectators.

Sport venues typically have crowds seated or standing in close proximity, whether for racing events (e.g. horse racing, car racing etc.) or for sports like football, baseball, basketball, hockey, golf, soccer or track and field. While masks can be (and should be) worn, social distancing is difficult. In a preferred embodiment, the present invention contemplates aerosolized interferon administration to participants and spectators prior to (or shortly thereafter) entering the venue (including self-administration).

Sport venues vary in the number of people in attendance. For example, NCAA Division I ice hockey arenas have capacities typically between 1000 and 8000 people (although a few have capacities of 15,000 or more). To take an example, Yost Ice Arena in Ann Arbor has a capacity of around 6000 people. Assuming 20 turnstiles and a throughput at each turnstile of approximately 5 people per minute, 6000 people can enter the arena in approximately 60 minutes. The present invention contemplates interferon administered through inhalation via an aerosol spray, mist, or powder that takes place at or near the turnstile (or other type of gateway), i.e. just prior to the person coming through the turnstile, or as the person is coming through the turnstile, or just after the person has come through the turnstile. Because of the ease of administration through inhalation, the present invention contemplates that this will slow the throughput at each turnstile to approximately 2-3 people per minute. Thus, assuming 20 turnstiles, 6000 people could enter the arena in approximately 150 or 100 minutes, respectively. In one embodiment, the stadium or arena adds turnstiles (or other types of gateways) in order to accommodate this additional time needed for interferon administration to the spectators entering the venue.

Professional sports venues have typically greater capacities. For example, Gillette Stadium in Foxboro, Mass. has a capacity of approximately 65,000. Assuming 100 turnstiles and a throughput at each turnstile of approximately 5 people per minute, 65,000 people can enter the stadium in approximately 130 minutes. The present invention contemplates interferon administered through inhalation via an aerosol spray, mist, or powder that takes place at or near the turnstile (or other type of gateway). Because of the ease of administration through inhalation, the present invention contemplates that this will slow the throughput at each turnstile to approximately 2-3 people per minute. Thus, assuming 100 turnstiles, 65,000 people could enter the stadium in approximately 325 or 217 minutes, respectively. In one embodiment, the stadium or arena adds turnstiles (or other types of gateways) in order to accommodate this additional time needed for interferon administration. Games could be scheduled for late in the day (4 PM, 5 PM, 6 PM, 7 PM etc.) to permit the processing of people for 3.5 to 5.5 hours. Moreover, there is a tradition of “tail gaiting” at football games, whereby a portion of the fans show up a number of hours before the start of the game. Stations (e.g. booths, tents, etc.) could be set up in the parking lot of football stadiums for these tail gaiting people in order to start the process of interferon administration earlier in the day. Finally, since NFL games are (for the most part) done on Sundays, the stadium could offer on-site interferon administration the day before the game. A fan given interferon the day before the game could be given an identifying credential (e.g. a badge, venue-created photo identifications (ID) and the like) at the site of administration or at a credentialing office at the venue, thereby indicating that the fan can now take his or her ticket to the entrance and gain admittance the following day. Each credential should be thoroughly checked prior to granting access to ensure compliance.

Where the capacity of the venue is 2000 or fewer, administration could be performed in places other than at the turnstile. For example, in one embodiment, booths, tents or the like are set up outside stadiums, arenas and the like, so that fans seeking entry can be administered aerosolized interferon, at which point such a fan could be given an identifying credential (e.g. a badge, venue-created photo identifications (ID) and the like) at the site of administration or at a credentialing office at the venue, thereby indicating that the fan can now take his or her ticket to the entrance and gain admittance. Each credential should be thoroughly checked prior to granting access. This can be done on game day but could also be done on a day prior to game day. In one embodiment, the person receiving the interferon is first tested for infection. In one embodiment, the person receiving the interferon is first tested for an elevated temperature. Each of these steps, however, adds time to the process that must be factored into an efficient entry to the venue or facility.

It is contemplated that the aerosolized interferon will provide the spectator a measure of protection against infection from fellow fans seated nearby or otherwise contacted during the event. Again, it is not required that aerosolized interferon administration provide 100% protection, i.e. such that no infections arise from the sporting event. It is sufficient that aerosolized interferon administration provide sufficient protection such that 1% of the attendees or less, more preferably 0.5% or less, still more preferably 0.1% or less, still more preferably 0.05% or less, and most preferably 0.01% or less end up infected (e.g. fewer infections than there would have been without the administration of aerosolized interferon). As noted above, even where there is infection, it is expected to be mild where the person has received interferon.

To complete the protective environment for such sports venues, the present invention contemplates aerosolized interferon administration for all of the officials, players, coaches, assistants and staff, as well as all of the arena or stadium staff (e.g., maintenance, engineering, administration), including all concession stand workers. All contractors (including news media, television, radio announcers) should also be administered aerosolized interferon prior to (or shortly after) entering the venue. Indeed, anyone and everyone in the arena and stadium should be administered aerosolized interferon down to the last cheerleader, the last marching band member and mascot. In one embodiment, interferon is administered to these people even though they have tested negative or would test negative if tested for the virus.

In very large stadiums or arenas (e.g. with a capacity for 50,000 or more people), the logistics of administering aerosolized interferon to so many people need to be taken into account. Each site around (or in) the stadium, whether a booth, tent, stall, stand, kiosk, table, window or counter, can only administer at a reasonable rate. Therefore, when calculating the number of sites required to get the job done, one needs to factor in the expected attendance. This is particularly true if the administrating is done on game day. In one embodiment, prior to administrating aerosolized interferon, the person coming to the site will first i) disinfect their hands, ii) present an ID, and iii) have their temperature taken. In one embodiment, persons with elevated temperatures are turned away. In one embodiment, the person will have their picture taken for their venue-issued credential. In one embodiment, the person will then be given aerosolized interferon. Following administration, the person will be given their venue-issued credential. Depending on the embodiment (and the number of the above-noted steps), this will take between 2 and 5 minutes per person. Assuming 3 minutes per person, each site will process approximately 20 people/hr. One hundred such sites will process 2000 people/hr.

It is not intended that the present invention be limited to administration of aerosolized interferon to only those at risk of being exposed to virus. In one embodiment, the present invention contemplates aerosolized interferon administration to people who have not been tested for COVID-19. Such untested people may not yet be infected (and therefore be only at risk of infection); however, such untested people may be infected but not know it (e.g. they have no symptoms yet, or their symptoms are so mild they are unaware of the infection). It is contemplated that there are benefits to administering aerosolized interferon to such infected individuals. First, the aerosolized interferon may help that infected individual fight off the infection (e.g. fight it off without it developing more severe symptoms, or fight it off more quickly than without aerosolized interferon). After all, the coronavirus known as SARS was shown to actively suppress host innate immune functions, including type I IFN expression, in infected cells. See Narayanan et al., “Severe acute respiratory syndrome coronavirus nsp1 suppresses host gene expression, including that of type I interferon, in infected cells,” J. Virology 2008 May; 82 (9):4471-9. Second, there is the likely benefit to people around such an (unknowingly) infected person, i.e. the administration of aerosolized interferon may result in the death of the virus in the person (e.g. directly or indirectly through the activation of the immune system), thereby lowering the amount of virus available to infect others, thereby reducing the spread of infection.

Early in 2021, the main concern was the Alpha variant, a variant originally identified in the U.K., which seemed to spread more easily than the original “wild type” coronavirus. The SARS-CoV-2 B.1.617 variant, also called the “Delta variant” was identified in October 2020 in India. It has since become dominant in some Indian regions and UK and further spread to many countries in 2021. The lineage includes three main subtypes (B1.617.1, B.1.617.2 and B.1.617.3), harbouring diverse Spike mutations in the N-terminal domain (NTD) and the receptor binding domain (RBD) which may increase their immune evasion potential. The Delta variant is believed to spread faster than other variants. A variant like alpha or delta is distinguished by changes to its genetic code that have effectively altered the virus's behavior. Delta has more than 20 differences in its genetic makeup, some of which may make it easier for the spike protein to bind to human cells and help the virus evade antibodies.

The Delta Variant has been found to be resistant to neutralization by some anti-NTD and anti-RBD mAbs including Bamlanivimab, which were impaired in binding to the Spike protein. Sera from convalescent patients collected up to 12 months post symptoms were 4-fold less potent against the Delta variant, relative to variant Alpha (B.1.1.7). Sera from individuals having received one dose of Pfizer or AstraZeneca vaccines barely inhibited variant Delta.

Administration of two doses generated a neutralizing response in 95% of individuals, but with titers 3- to 5-fold lower against Delta than Alpha. Thus, variant Delta spread is associated with an escape to antibodies targeting non-RBD and RBD Spike epitopes. Indeed, in late July 2021, fully vaccinated people got infected with the Delta variant of COVID-19 at a public event in a town on Cape Cod in Massachusetts. Unfortunately, vaccinated individuals had a similar amount of virus present as the unvaccinated, suggesting that, unlike with other variants, vaccinated people infected with the Delta variant can transmit the virus. During the summer of 2021, Australia went through a cycle of stop-start lockdowns in several cities after the emergence of the fast-moving Delta strain, and such restrictions are likely to persist into the Fall until the country reaches a much higher level of vaccination coverage.

The Delta variant is believed to spread faster than other strains of the coronavirus because it makes more copies of itself inside our bodies more quickly than other strains of the coronavirus. In research posted online in July of 2021, Chinese scientists detected Delta viral loads that were about 1,260 times higher than earlier strains on initial positive tests. They compared 62 Delta cases from 2021 with 63 cases from the early epidemic wave in 2020. Moreover, the amount of time it took quarantined people to test positive for Covid-19 on PCR also shortened—from about six days with the earlier infections to four days with the Delta variant.

The Delta variant is creating havoc in states where large portions of the population are unvaccinated. In Missouri, the Delta variant accounts for 95 percent of new cases. It is spreading easily in many states because people have stopped wearing their masks, crowded into indoor spaces, resumed travel, and resisted vaccinations. There is now the so-called “delta plus” variant, which is causing alarm among governments and health officials? First identified in Europe in March of 2021, the variant is also known as B. 1.617.2.1 or AY.1. The “plus” of the variant's name refers to its K417N spike protein mutation, which was also found in some substrains of the alpha variant. The present invention contemplates using interferon (as described herein) against the “plus” variant as well.

The Delta variant is devastating parts of Asia in July of 2021. Asia is home to many of the world's 1.7 million commercial seafarers—has prompted many nations to cut off land access to visiting crews, in some cases even for medical treatment. Just 2.5% of seafarers—one in every 40—have been vaccinated. There are about 100,000 seafarers stranded at sea beyond their regular stints of typically 3-9 months, according to the International Chamber of Shipping (ICS), many without even a day's break on land. Another 100,000 are stuck on shore, unable to board the ships they need to earn a living on. Since these ships transport around 90% of the world's trade, the deepening crisis poses a major threat to the supply chains relied on for everything from oil and iron to food and electronics. In one embodiment, the present invention contemplates administering an interferon (INF), whether Type I (e.g. interferon-alpha, interferon-beta, etc.), Type II (e.g. interferon-gamma) and/or Type III (e.g. interferon-lamda), for inhibiting and/or preventing coronavirus in such commercial seafarers, both at sea and on land.

In one embodiment, the present invention contemplates administering an interferon (INF), whether Type I (e.g. interferon-alpha, interferon-beta, etc.), Type II (e.g. interferon-gamma) and/or Type III (e.g. interferon-lamda), for inhibiting and/or preventing coronavirus, and in particular to protect against the Delta variant. Such individuals can be vaccinated or unvaccinated. Thus, such an interferon can be administered (e.g. by inhalation) to a vaccinated person (i.e. vaccinated against the original Covid-19) to reduce the risk of infection by the vaccinated person to new coronavirus variants, including but not limited to the Delta variant and the Delta “plus” variant.

In one embodiment, the present invention contemplates administering an interferon (INF), whether Type I (e.g. interferon-alpha, interferon-beta, etc.), Type II (e.g. interferon-gamma) and/or Type III (e.g. interferon-lamda), for inhibiting and/or preventing coronavirus infection in vaccinated or unvaccinated people in a state (e.g. Alabama, Missouri, etc.) or a country (e.g. India, Brazil, etc.) where the Delta variant has been detected and/or where the Delta variant (or other variant) is found responsible for 80% or more (or 90% or more) of the coronavirus hospitalizations.

### DETAILED DESCRIPTION

Type I interferons are a family of closely related glycoproteins comprised of thirteen IFN-α subtypes as well as IFN-β, IFN-K, IFN-τ and IFN-ω. The different human IFN-α subtypes have been identified by analysis of human cDNA libraries and by protein analysis of the IFNs produced by stimulated lymphoblastoid cells; the reasons for their heterogeneity remain unclear. Early studies indicated that all subtypes bind the same receptor from which it was inferred that they must elicit identical responses. Subsequently, comparative studies of both purified and recombinant subtypes revealed a spectrum of anti-viral, antiproliferative and immunomodulatory responses.

Interferon-alpha (IFN-alpha) has been used clinically and commercially (e.g., RoferonA™, IntronA™, Pegasys™, Peglntron™ etc) to successfully treat various cancers, including, e.g., malignant melanoma, hairy cell leukemia, non-Hodgkin's lymphoma, AIDS-related Kaposi's sarcoma, as well as infectious diseases, such as severe acute respiratory syndrome (SARS), chronic Hepatitis B, and chronic Hepatitis C.

IFN-alpha is a type I interferon, which binds to the IFN-alpha receptor. IFN-alpha is one of the earliest cytokines released by antigen presenting cells as part of the innate immune response. It is directly responsible for NK and T cell responsiveness, which drives the subsequent immune response. Because of the early response of IFN-alpha in the immune cascade, its primary role is suggested to be to induce a priming state during the initial response to infection.

IFN-beta for use in accordance with the invention will be understood to refer to any form or analogue or synthetic non-natural derivative of IFN-beta that retains the required biological activity of native IFN-beta (although the exact level of the native compound is not needed). It may preferably be a recombinant IFN-beta, e.g. a commercially available IFN-beta including but not limited to recombinant IFN-beta 1a, IFN-beta 1b, Betaseron™, Betaferon™, Avonex™, Rebif™ and formulations manufactured by Rentschler GmbH or any other manufacturer.

Interferon lambda (IFNλ) is a group of cytokines that belong to the IL-10 family. They exhibit antiviral activities against certain viruses during infection of the liver and mucosal tissues. The identification of IFN-λs 1, 2, 3 & 4 (termed as type III IFNs) has revealed that the antiviral immune response to viruses contains more components than the type I IFNs that have been known for more than 50 years. IFN-λs are IFN-λ1 (IL-29), IFN-λ2 (IL-28a), IFN-λ3 (IL-28b) and IFN-λ4, which resembles IFN-λ3. IFN-λs have type I-IFN-like immune responses and biological activities. J. Zhou et al., “Type III Interferons in Viral Infection and Antiviral Immunity,” Cell Physiol Biochem 2018; 51:173-185. They are distinct from both type I and type II IFNs for a number of reasons, including the fact that they signal through a heterodimeric receptor complex that is different from the receptors used by type I or type II IFNs. Although type I IFNs (IFN-α/β) and type III IFNs (IFN-λ) signal via distinct receptor complexes, they activate the same intracellular signaling pathway and many of the same biological activities, including antiviral activity, in a wide variety of target cells. IFN-lamda, for use in accordance with the invention may be any form or analogue or synthetic non-natural derivative of IFN-lamda that retains the required biological activity (similar activity) of a native form, preferably a recombinant IFN-lamda. One or more polypeptides selected from recombinant versions or analogues of these may be employed as detailed in WO 2007/029041, the contents of which are hereby incorporated by reference.

Interferon-gamma is a pleiotropic cytokine that has specific immune-modulating effects, e.g. activation of macrophages, enhanced release of oxygen radicals, microbial killing, enhanced expression of MHC Class II molecules, anti-viral effects, induction of the inducible nitric oxide synthase gene and release of NO, chemotactic factors to recruit and activate immune effector cells, down regulation of transferrin receptors limiting microbial access to iron necessary for survival of intracellular pathogens, etc. Genetically engineered mice that lack interferon-gamma or its receptor are extremely susceptible to mycobacterial infection. Recombinant interferon-gamma is commercially available as ACTIMMUNE™ from InterMune, Brisbane, Calif.

No systemic side effects are expected. Indeed, Jaffe found that aerosolized INF-gamma. given to normal subjects was safe, without systemic side effects, and was able to activate alveolar macrophages and not PBMC, as opposed to parenterally delivered INF-gamma, the effects of which could only be noted in the peripheral blood. See Jaffe et al., (1991) J Clin Invest 88 (1): 297-302.

In more specific embodiments, aerosolized interferon is administered in doses ranging from 0.1 to 1000 micrograms (ug), preferably about 1 to 10 ug, 10 to 250 ug or 75 to 500 ug or 100 to 250 ug, preferably given in a nebulizer. Since systemic administration is not contemplated, the present invention contemplates, in one embodiment, giving every adult the same dose by inhalation (regardless of their weight). As noted above, it is preferred that the interferon be delivered in a single administration e.g. prior to an event (such as a team sport). However, athletes competing regularly may need interferon by inhalation from one to ten times per week, preferably about two, three, four or five times per week. In another embodiment, a dose of 100 to 500 ug is given in a nebulizer to members of sport teams three times per week. Lower doses may be given depending on the efficiency of the nebulizer. When given more than one time per week, the aerosolized interferon may be titrated to ensure no undesirable effects are experienced by these people.

In a preferred embodiment, the aerosolized interferon is given once to a person just prior to entering a structure, vehicle or situation involving close contact with multiple people or crowds, including but not limited to a sporting event, a movie or theater event, travel (whether on plane, train, boat or bus), or school.

It is not intended that the present invention be limited by the device by which aerosolized interferon is administered. It is an object of the present invention to deliver the interferon via the oral, nasal and/or pulmonary route of administration. Contemplated for use in the practice of this invention are a wide range of mechanical devices designed for delivery of aerosolized drugs, including but not limited to nebulizers, metered dose inhalers, and powder inhalers, all of which are familiar to those skilled in the art. For example, a metered dose inhaler (MDI) is a small handheld device that disperses medication into the airways via an aerosol spray or mist through the activation of a propellant. A measured dose of the drug is delivered with each push of a canister, and dosing is usually achieved with one or two puffs.

Some specific examples of commercially available devices suitable for the practice of this invention are the ULTRAVENT™ nebulizer, manufactured by Mallinckrodt, Inc., St. Louis, Mo.; the ACORN II™ nebulizer, manufactured by Marquest Medical Products, Englewood, Colo.; the VENTOLIN™ metered dose inhaler, manufactured by Glaxo Inc., Research Triangle Park, N.C.; and the SPINHALER™ powder inhaler, manufactured by Fisons Corp., Bedford, Mass., MISTYNEB™, manufactured by Allegiance, McGraw Park, Ill.; AEROECLIPSE™, manufactured by Trudell Medical International, Canada, and the I-NEB™ manufactured by Philips Respironics.

All such devices require the use of formulations suitable for the dispensing of protein. This can be as simple as sterile water or a saline solution. Typically, each formulation is specific to the type of device employed and may involve the use of an appropriate propellant material, in addition to the usual diluents, adjuvants and/or carriers. Also, the use of liposomes, microcapsules or microspheres, inclusion complexes, or other types of carriers is contemplated. Chemically modified protein may also be prepared in different formulations depending on the type of chemical modification or the type of device employed.

Formulations suitable for use with a nebulizer, either jet or ultrasonic, may typically comprise protein dissolved in water at a concentration of about 0.01 to 25 mg of biologically active protein per mL of solution. The formulation may also include a buffer and a simple sugar (e.g., for protein stabilization and regulation of osmotic pressure). The nebulizer formulation may also contain a surfactant, to reduce or prevent surface induced aggregation of the protein caused by atomization of the solution in forming the aerosol.

Formulations for use with a metered-dose inhaler device may generally comprise a finely divided powder containing the protein suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device may comprise a finely divided dry powder containing protein and may also include a bulking agent, such as lactose, sorbitol, sucrose, or mannitol in amounts which facilitate dispersal of the powder from the device, e.g., 50 to 90% by weight of the formulation. The protein should most advantageously be prepared in particulate form with an average particle size of less than 10 microns, most preferably 0.5 to 5 microns, for most effective delivery to the distal lung (if that is desired). Deep inspiration may, when compared with standard breathing, increase deposition efficiency in the lung periphery.

Vaccines available include Pfizer's vaccine and Moderna's vaccine (as well as some others). Researchers in the United States have conducted a study showing that the coronavirus disease 2019 (COVID-19) vaccines developed by Pfizer-BioNTech and Moderna generate a coordinated adaptive immune response that is capable of eliciting recall responses to future infection with severe acute respiratory syndrome coronavirus 2 (SARS-CoV-2). The team from the University of California found that immunization with either Pfizer-BioNTech's BNT162b2 or Moderna's mRNA-1273 vaccine-induced enrichment of spike-specific B cells.

The J&J vaccine TNJ-78436735 was approved and then briefly stopped. The Centers for Disease Control and Prevention (CDC) and the US Food and Drug Administration (FDA) recommended that use of (J&J/Janssen) COVID-19 Vaccine resume in the United States, effective Apr. 23, 2021.

A vaccinated person can be a person vaccinated with either Pfizer's vaccine or Moderna's vaccine. Alternatively, a vaccinated person can be person vaccinated with the J&J or AstraZeneca vaccine. Vaxzevria (formerly COVID-19 Vaccine AstraZeneca) is authorized in the EU to prevent COVID-19. An unvaccinated person is a person who has been vaccinated with none of these vaccines.

### DESCRIPTION OF SOME EMBODIMENTS

Interferons are a family of naturally-occurring proteins that are produced by cells of the immune system. Four classes of interferons have been identified, alpha, beta, lambda and gamma. Each class has different effects though their activities overlap. Together, the interferons direct the immune system's attack on viruses, bacteria, tumors and other foreign substances that may invade the body. Once interferons have detected and attacked a foreign substance, they alter it by slowing, blocking, or changing its growth or function.

Aspects of the present specification disclose, in part, a pharmaceutical composition. A pharmaceutical composition disclosed herein is useful for human medical and veterinary applications. A pharmaceutical composition may be administered to an individual alone, or in combination with other supplementary active ingredients, agents, drugs or hormones.

A pharmaceutical composition disclosed herein (whether as an aerosol, mist or droplets) may include a pharmaceutically acceptable carrier that facilitates stabilizing and/or processing of an active ingredient into pharmaceutically acceptable compositions. As used herein, the term “pharmacologically acceptable carrier” is synonymous with “pharmacological carrier” and means any carrier that has substantially no long term or permanent detrimental effect when administered and encompasses terms such as “pharmacologically acceptable vehicle,” “stabilizer,” “diluent,” “additive,” “auxiliary” or “excipient.” Such a carrier generally is mixed with an active compound or permitted to dilute or enclose the active compound. It is understood that the active ingredients can be soluble or can be delivered as a suspension, aerosol or mist in the desired carrier or diluent. Any of a variety of pharmaceutically acceptable carriers can be used including, without limitation, aqueous media such as, e.g., water, saline, glycine, hyaluronic acid and the like; carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7th ed. 1999); REMINGTON: THE SCIENCE AND PRACTICE OF PHARMACY (Alfonso R. Gennaro ed., Lippincott, Williams & Wilkins, 20th ed. 2000); Goodman & Gilman's The Pharmacological Basis of Therapeutics (Joel G. Hardman et al., eds., McGraw-Hill Professional, 10th ed. 2001); and Handbook of Pharmaceutical Excipients (Raymond C. Rowe et al., APhA Publications, 4th edition 2003). These protocols are routine procedures and any modifications are well within the scope of one skilled in the art and from the teaching herein.

A pharmaceutical composition disclosed herein may comprise a solvent in an amount sufficient to dissolve a compound such as an interferon disclosed herein. In other aspects of this embodiment, a pharmaceutical composition disclosed herein may comprise a solvent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects of this embodiment, a pharmaceutical composition disclosed herein may comprise a solvent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

In one embodiment, a solvent may comprise a pharmaceutically acceptable alcohol. As used herein, the term “alcohol” refers to an organic molecule comprising a hydroxyl functional group (—OH) bonded to a carbon atom, where the carbon atom is saturated. In other aspects of this embodiment, an alcohol may be, e.g., a primary alcohol, a secondary alcohol, or a tertiary alcohol. In other aspects of this embodiment, an alcohol may be, e.g., an acyclic alcohol, a monohydric alcohol, a polyhydric alcohol (also known as a polyol or sugar alcohol), an unsaturated aliphatic alcohol, an alicyclic alcohol, or a combination thereof. Examples of a monohydric alcohol include, without limitation, methanol, ethanol, propanol, butanol, pentanol, and 1-hexadecanol. Examples of a polyhydric alcohol include, without limitation, glycol, glycerol, arabitol, erythritol, xylitol, maltitol, sorbitol (gluctiol), mannitol, inositol, lactitol, galactitol (iditol), and isomalt. Examples of an unsaturated aliphatic alcohol include, without limitation, prop-2-ene-1-ol, 3,7-dimethylocta-2,6-dien-1-ol, and prop-2-in-1-ol. Examples of an alicyclic alcohol include, without limitation, cyclohexane-1,2,3,4,5,6-hexyl and 2-(2-propyl)-5-methyl-cyclohexane-1-ol.

In another embodiment, a solvent may comprise an ester of pharmaceutically acceptable alcohol and an acid. Suitable pharmaceutically acceptable alcohols include the ones disclosed herein. Suitable acids include, without limitation, acetic acid, butaric acid, and formic acid. An ester of an alcohol and an acid include, without limitation, methyl acetate, methyl buterate, methyl formate, ethyl acetate, ethyl buterate, ethyl formate, propyl acetate, propyl buterate, propyl formate, butyl acetate, butyl buterate, butyl formate, isobutyl acetate, isobutyl buterate, isobutyl formate, pentyl acetate, pentyl buterate, pentyl formate, and 1-hexadecyl acetate, 1-hexadecyl buterate, and 1-hexadecyl formate.

In another embodiment, a solvent may comprise a pharmaceutically acceptable polyethylene glycol (PEG) polymer. PEG polymers, also known as polyethylene oxide (PEO) polymers or polyoxyethylene (POE) polymers, are prepared by polymerization of ethylene oxide and are commercially available over a wide range of molecular weights from 100 g/mol to 10,000,000 g/mol. PEG polymers with a low molecular mass are liquids or low-melting solids, whereas PEG polymers of a higher molecular mass are solids. A PEG polymer include, without limitation, PEG 100, PEG 200, PEG 300, PEG 400, PEG 500, PEG 600, PEG 700, PEG 800, PEG 900, PEG 1000, PEG 1100, PEG 1200, PEG 1300, PEG 1400, PEG 1500, PEG 1600, PEG 1700, PEG 1800, PEG 1900, PEG 2000, PEG 2100, PEG 2200, PEG 2300, PEG 2400, PEG 2500, PEG 2600, PEG 2700, PEG 2800, PEG 2900, PEG 3000, PEG 3250, PEG 3350, PEG 3500, PEG 3750, PEG 4000, PEG 4250, PEG 4500, PEG 4750, PEG 5000, PEG 5500, PEG 6000, PEG 6500, PEG 7000, PEG 7500, PEG 8000, PEG 8500, PEG 9000, PEG 9500, PEG 10,000, PEG 11,000, PEG 12,000, PEG 13,000, PEG 14,000, PEG 15,000, PEG 16,000, PEG 17,000, PEG 18,000, PEG 19,000, or PEG 20,000.

In yet other aspects of this embodiment, a effective amount of a preventative compound such as an interferon disclosed herein generally is in the range of about 0.0001 mg/kg to about 100 mg/kg. In aspects of this embodiment, an effective amount of a preventative compound disclosed herein may be, e.g., at least 0.001 mg/kg/day, at least 0.01 mg/kg/day, at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, or at least 50 mg/kg/day.

Dosing can be single dosage (which is preferred) or cumulative (serial dosing). In a preferred embodiment, aerosolized interferon is given once as a pre-requisite to entry to a structure, vehicle or situation involving other people (including crowds of people, i.e. 20-100 people, 100-1000 people, and in the case of sporting events 10,000 to 100,000 people or more).

Above, it has been noted that combinations of interferons are contemplated. For example, a Type I interferon might be used together with a Type III interferon. However, it is not intended that the present invention be limited to such a combination. Mixtures and combinations within a Type of interferon are also contemplated. For example, in one embodiment, the present invention contemplates a method comprising administering via inhalation one type of IFNλ polypeptide or a mixture of IFNλ-1 and IFNλ-2, or a mixture of IFNλ-1 and IFNλ-3, or a mixture of IFNλ-2 and IFNλ-3, or a mixture of IFNλ-1, IFNλ-2 and IFNλ-3. By “IFNλ polypeptide” those polypeptides disclosed in GenBank accession numbers Q8IU54, Q8IZJ0, Q8IZI9, are meant to be included. Again, this is just an example, and other combinations are contemplated to be within the scope of the invention.

While natural or native interferon is preferred, other versions of interferons have been made over the years and been approved for human use. Thus, in some embodiment, a non-native interferon is contemplated. For example, recombinant interferon may be a slightly modified interferon in order to allow for better production.

1. A method of inhibiting coronavirus infection comprising administering interferon to at least a portion of the upper respiratory tract of a person at risk of being exposed to a coronavirus.

2. The method of claim 1, wherein said interferon is selected from the group consisting of a Type I interferon, a Type II interferon, a Type III interferon, or combinations thereof.

3. The method of claim 2, wherein said interferon is IFN-alpha.

4. The method of claim 2, wherein said interferon is IFN-beta.

5. The method of claim 2, wherein said interferon is IFN-lambda.

6. The method of claim 2, wherein said interferon is IFN-gamma.

7. The method of claim 1, wherein said administering is through inhalation of an aerosol, spray, mist, or powder comprising said interferon.

8. The method of claim 7, wherein said administering is done by a health professional.

9. The method of claim 7, wherein said administering is self-administration.

10. The method of claim 1, wherein the person is elderly.

11. The method of claim 1, wherein the person is a patient or worker in a nursing home.

12. The method of claim 1, wherein the person is a student or teacher in a school.

13. The method of claim 1, wherein the person is a worker in a meatpacking and/or food processing facility.

14. The method of claim 1, wherein the person is a member of a sports team.

15. The method of claim 1, wherein the person is a hospital worker.

16. The method of claim 15, wherein said hospital has patients who have tested positive for the Delta variant of COVID-19.

17. The method of claim 15, wherein said hospital worker has been vaccinated.

18. The method of claim 1, wherein the person is a transportation worker.

19. The method of claim 1, wherein the person is spectator at an event.

20. The method of claim 2, wherein more than one type of interferon is administered simultaneously, separately or sequentially.

21. The method of claim 1, wherein the interferon is administered by means of an aerosol nebuliser.

22. The method of claim 1, wherein between 1 and 1,000 micrograms of interferon is administered in a single administration.

23. The method of claim 22, wherein said single administration takes 1 minute or less.

24. A method for inhibiting viral infection of a person, comprising administering 1 to 1,000 micrograms of an interferon to said person via inhalation prior to close contact with multiple people or crowds.

25. The method of claim 24, wherein said interferon is selected from the group consisting of a Type I interferon, a Type II interferon, a Type III interferon, or combinations thereof.

26. The method of claim 24, wherein said administering is through inhalation of an aerosol, spray, mist, or powder comprising said interferon.

27. The method of claim 24, wherein said person is a member of a sports team and said aerosolized interferon is administered prior to practice or a game with other members of a sports team.

28. The method of claim 24, wherein said interferon is aerosolized.

29. The method of claim 28, wherein said aerosolized interferon contacts at least the nasal mucosa of said person.

30. The method of claim 24, wherein said viral infection is a coronavirus infection.

31. The method of claim 28, wherein a single administration of less than 100 ug of aerosolized interferon is administered using a nebulizer.

32. The method of claim 28, wherein said administering results in a portion of said aerosolized interferon depositing in the lungs of said person.

33. A method for inhibiting viral infection of a sports team, comprising administering 1 to 1,000 micrograms of an interferon to at least all of the participating members of a first sports team via inhalation prior to practice or a game.

34. The method of claim 33, wherein said viral infection is a coronavirus infection.
